(19) United States
(12) Patent Application Publication
(10) Pub. No.: US 2021/0244409 A1
(43) Pub. Date: Aug. 12, 2021

(54) SUTURE MATERIAL DEVELOPED FOR END-TO-END ANASTOMOSIS

(71) Applicant: Ali Engin ULUSAL, Balikesir (TR)

(72) Inventor: Ali Engin ULUSAL, Balikesir (TR)

(21) Appl. No.: 17/271,283

(22) PCT Filed: Sep. 18, 2018
(86) PCT No.: PCT/TR2018/050502

§ 371 Date: Feb. 25, 2021

(30) Foreign Application Priority Data
Aug. 29, 2018 (TR) 2018/12305

(51) Int. Cl. A61B 17/06; A61B 17/11

(52) U.S. Cl. CPC A61B 17/06166; A61B 2017/1132; A61B 2017/1107; A61B 17/11

(57) ABSTRACT

A suture material to be placed in blood vessels which is developed for end-to-end anastomosis of blood vessels with a lumen in microsurgery in a way to enable end-to-end suturing of blood vessels while at the same time increasing the success of vessel sutures and contributing to keeping the lumen open, wherein the straight wires form the body structure such that a rhombic lattice structure will be obtained, and then the oval wires are passed through the circles positioned at the end portions and knitted by the practitioner.

### TECHNICAL FIELD

The invention relates to a suture material developed for end-to-end arterial anastomosis in microsurgery.

The invention particularly relates to a suture material which is placed in blood vessels and formed such that the straight wires will form the body structure in a way to obtain a rhombic lattice structure, and then the oval wires will be passed through the circles positioned at the end portions and knitted, in order to ensure end-to-end suturing of luminal blood vessels while at the same time increasing the success of vessel sutures and contributing to keeping the lumen open.

### STATE OF THE ART

Microsurgery is a surgical method performed under surgical microscope using very fine tipped (high precision) specialized instruments. These surgical operations are performed by experienced teams trained for microsurgery. Thus, sometimes fine veins as small as 1 mm diameter can be sutured together.

Microsurgical anastomosis is based on the principle that the positional relationship of vessels on which anastomosis is performed is taken into consideration, and then the vessels are sutured together after being approximated in a way to oppose one another with interrupted sutures. End-to-end anastomosis is a frequently used technique in clinical applications.

Arterial anastomosis performed today involves suturing with nylon suture thread of 8/0, 9/0 and 10/0, from thick to thin. Although suturing is performed by surgeons particularly trained for such techniques, there is a high likelihood of anastomosis failure. Here, it is of utmost importance to place sutures at appropriate intervals. Atraumatic method and meticulousness are required; in particular, care must be taken not to damage tunica intima. Otherwise, vascular structure may be damaged or arterial spasm may occur at this stage. Spasm refers to contraction of blood vessel wall reflexively and narrowing of the lumen and it may thus cause the blood flow to stop. This anastomosis technique carried out by classical methods involves such complications and may eventually cause clogging of anastomosis and the blood flow to stop as well as necrosis of the transplanted tissue, and thus leading to the failure of the surgery.

The problem of clogging in vessel sutures is quite common. Clogging may result from a technical error, or may as well be due to arterial spasm or arteriosclerosis. The suture material to be used does not have any contribution to the vessel remaining open; and occasionally the suture itself may block the lumen. In some cases, however, the suture may pass through the opposite wall of the vessel and may thus result in complete blockage of vessel lumen. All of the drawbacks mentioned above cause a decrease in the likelihood of the success of surgery. In addition to these, the likelihood of success of anastomosis becomes low due either to vein walls being thinner and finer and or the blood flow being not as fast and pressurized as the artery.

As a result, there exists a need for novelty in the related technical field in a way to increase the success of vessel sutures, contribute to the vessel lumen remaining open, eliminate the aforementioned drawbacks and overcome the deficiencies.

### OBJECTS OF THE INVENTION

The present disclosure relates to a suture material developed for end-to-end anastomosis which meets all the requirements mentioned above, eliminates all of the disadvantages and provides some additional advantages.

The main object of the invention is to increase the success of vessel sutures and contribute to keeping the vessel lumen open.

Another object of the invention is to provide a system that can be readily applied even in very small vessels.

Yet another object of the invention is to simplify technical challenges of anastomosis procedure.

Still another object of the invention is to prevent blockage in suture line and safely continue blood flow in the vessel, after suturing the vessels.

Another object of the invention is to make such procedures widespread thanks to the newly developed easy and practical application method.

Another object of the invention is to shorten training periods of microsurgery and to facilitate it.

Yet another object of the invention is to shorten the duration of surgery.

And another object of the invention is to achieve more successful results, particularly while suturing veins.

The invention, with a view to achieving the objects mentioned above, is a suture material to be placed in vessels which is developed for end-to-end anastomosis of luminal blood vessels in microsurgery in a way to enable end-to-end suturing of blood vessels while at the same time increasing the success of vessel sutures and contributing to keeping the lumen open, characterized in that:
straight wires form the body structure such that a rhombic lattice structure will be obtained, and then the oval wires are passed through the circles positioned at the end portions and knitted by the practitioner.

The invention, with a view to achieving the objects mentioned above, is an operating method of suture materials, characterized in comprising the process steps of:
obtaining the suture material as a result of the straight wires forming the body structure such that a rhombic lattice structure will be obtained, and then the oval wires being passed through the circles positioned at the end portions and knitted,
placing a intravascular end of the suture material into the vessels on which end-to-end anastomosis will be performed by way of an applicator or catheter such that it will remain inside the lumen,
passing the straight wire which has the closest position to the blood vessel wall at a extravascular end of the suture material, through the circle of the proximate oval wire first,
removing the straight wire, which is passed through the circle of the oval wire, by piercing the vessel wall,
joining the ends of the vessels to be sutured such that their ends will face one another, and
suturing together the suture material, which is now placed in both blood vessels, as a result of knotting the straight wires removed out of the blood vessel wall.

The structural and characteristic features and all advantages of the invention will be understood more clearly by referring to the following detailed description; therefore, the evaluation needs to be done by taking this detailed description into consideration.

### FIGURES FOR A BETTER UNDERSTANDING OF THE INVENTION

FIG. 1: Perspective view of the suture material.

FIG. 2: Outside-in view of the suture material.

FIG. 3: Top view of the suture material when placed in the vessel.

FIG. 4: Detailed view of the suture material when placed in the vessel.

FIG. 5: Overall view showing the placement of vessels which comprise suture material in a way to one another.

FIG. 6: Overall view of the vessels the end-to-end anastomosis of which is conducted by means of the suture material.

The drawings do not necessarily need to be scaled and the details that are not necessary to understand the present invention may have been omitted. Furthermore, the elements which are at least substantially identical or have at least substantially identical functions are indicated with the same numeral.

### DESCRIPTION OF PART REFERENCES

10. Suture material

11. Straight wire

12. Oval wire

121. Circle

20. Blood vessel

21. Blood vessel wall

22. Lumen

A. Lattice structure

D. Knot

X. Intravascular end

Y. Extravascular end

### DETAILED DESCRIPTION OF THE INVENTION

In this detailed description, the preferred embodiments of the suture material (10) developed for end-to-end anastomosis of blood vessels (20) with lumen (22) in microsurgery according to the invention will be explained in order for the invention to be better understood, without any limitation.

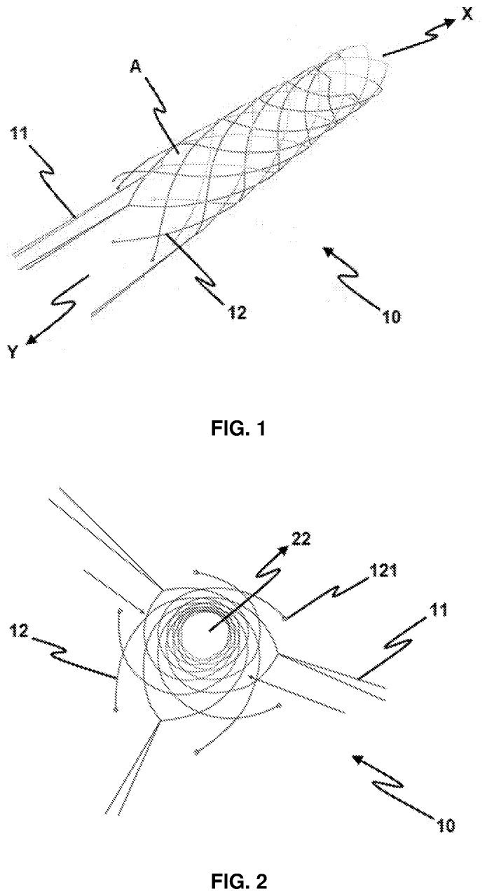

FIG. 1 illustrates the perspective view of the suture material (10). In general, the suture material (10) which is placed in blood vessels (20) in order to ensure end-to-end suturing of blood vessels (20) with lumen (22) while at the same time increasing the success of vessel sutures and contributing to keeping the lumen (22) open, is formed such that the straight wires (11) will form the body structure in a way to obtain a rhombic lattice structure (A), and then the oval wires (12) will be passed through the circles (121) positioned at the end portions and knitted by the practitioner.

The suture material (10) has a cylindrical form and has the appearance of a wire mesh thanks to a plurality of rhombic lattice structures (A) that it contains. Hence, the suture material (10) encloses the blood vessel walls (21) from inside and ensures that the lumen (22) remains hollow. Therefore, the lumen (22) is enabled to remain open.

In the preferred embodiment of the invention, the suture material (10) may be coated with antithrombotic or antibiotic medicaments.

At least six straight wires (11) and at least six oval wires (12) are used while forming the suture material (10). The straight wires (11) and oval wires (12) can be made of metal, metal alloys, polymer or composite material.

The intravascular end (X) of the suture material (10) is the portion which remains inside the vessel (20), i.e. within lumen (22). In the intravascular end (X), the ends of the straight wires (11) and oval wires (12) have a closed structure due to knitting. The extravascular end (Y), on the other hand, consists of straight wires (11) the knitting of which is completed and left in sets of two in a long and respective manner and oval wires (12) which are left in short form such that their circles (121) will be hollow. FIG. 2 shows outside-in view of the suture material (10).

The Operating Principle of the Suture Material (10) According to the Invention is as Follows:

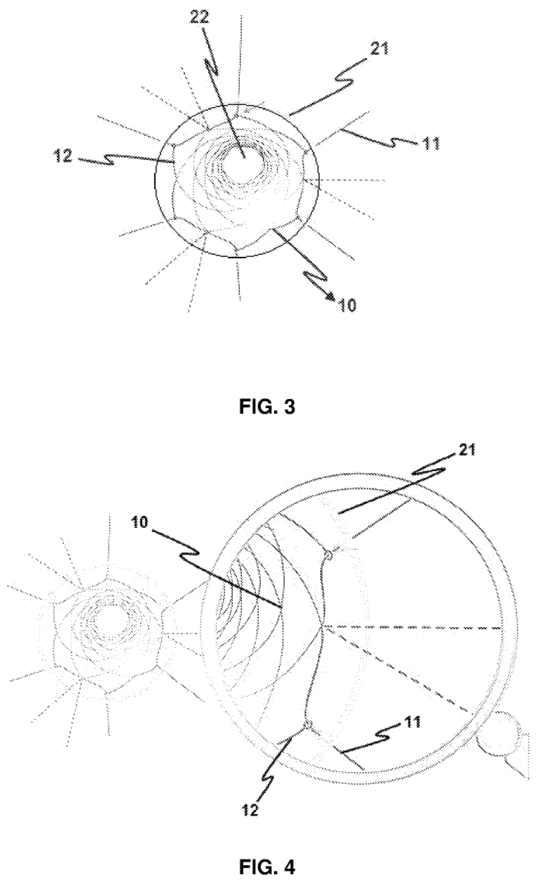

The intravascular end (X) of the suture material (10) which is formed as a result of the straight wires (11) forming the body structure in a way to obtain a rhombic lattice structure (A), and then the oval wires (12) being passed through the circles (121) positioned at the end portions and knitted, is placed in the blood vessel (20) on which end-to-end anastomosis is to be performed such that it will remain inside the lumen (22) by means of an applicator or a catheter. The straight wire (11) which has the closest position to the blood vessel wall (21) at the extravascular end (Y) of the suture material (10) is first passed through the circle (121) of the proximate oval wire (12), and immediately thereafter it is taken out by piercing the blood vessel wall (21). The same is also applied to the other blood vessel (20) on which end-to-end anastomosis will be performed. FIG. 3 shows the top view of the suture material (10) when placed in the blood vessel (20) whilst FIG. 4 illustrates the detailed view thereof.

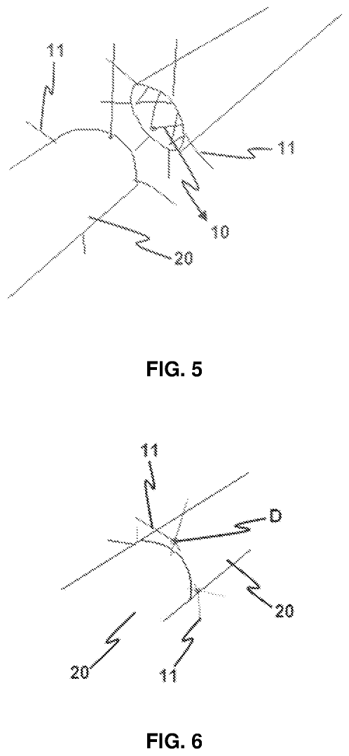

FIG. 5 shows the overall view showing the placement of blood vessels (20) which comprise suture material (10) in a way to oppose one another. The ends of the blood vessels (20) to be sutured are joined together such that their ends will face one another. The suture material (10), which is now placed in both blood vessels (20), is sutured together as a result of knotting (D) the straight wires (11) removed out of the blood vessel wall (21). FIG. 6 shows the overall view of the blood vessels (20) the end-to-end anastomosis of which is conducted by means of the suture material (10).

It is envisaged that the suture material (10) according to the invention will result in remarkable improvements in medical field, especially in the area of organ transplantation, as well as contributing to the related field such that success rates of organ transplantation surgeries will be increased. Moreover, it is expected to be widely applied in microsurgery and enable the surgeons to be more confident of themselves and of their surgery.

1. A suture material to be placed in blood vessels which is developed for end-to-end anastomosis of blood vessels with a lumen in microsurgery in a way to enable end-to-end suturing of blood vessels while at the same time increasing the success of vessel sutures and contributing to keeping the lumen open, characterized in that:
straight wires form the body structure such that a rhombic lattice structure will be obtained, and then oval wires are passed through circles positioned at the end portions and knitted by the practitioner.

2. The suture material of claim 1, wherein the material is coated with antithrombotic or antibiotic medicaments.

3. The suture material of claim 1, wherein said straight wires and oval wires are made of metal, metal alloys, polymer or composite material.

4. The operating method of the suture material, characterized in comprising the process steps of:
obtaining the suture material as a result of straight wires forming the body structure such that a rhombic lattice structure will be obtained, and then oval wires being passed through circles positioned at the end portions and knitted,
placing a intravascular end of the suture material into blood vessels on which end-to-end anastomosis will be performed by way of an applicator or catheter such that it will remain inside the lumen,
passing the straight wire which has the closest position to the blood vessel wall at a extravascular end of the suture material, through the circle of the proximate oval wire first,
removing the straight wire, which is passed through the circle of the oval wire, by piercing the vessel wall,
joining the ends of the blood vessels to be sutured such that their ends will face one another, and
suturing together the suture material, which is now placed in both blood vessels, as a result of knotting the straight wires removed out of the blood vessel wall.
